(19) United States
(12) Patent Application Publication
(10) Pub. No.: US 2013/0172802 A1
(43) Pub. Date: Jul. 4, 2013

(54) Moisture Removal Device and Method for Bariatric Skin Fold

(71) Applicants: Matthew Cavanaugh, San Antonio, TX (US); Matthew Pickering, San Antonio, TX (US); Eric Tan, San Antonio, TX (US); Manuel Torres, San Antonio, TX (US)

(72) Inventors: Matthew Cavanaugh, San Antonio, TX (US); Matthew Pickering, San Antonio, TX (US); Eric Tan, San Antonio, TX (US); Manuel Torres, San Antonio, TX (US)

(21) Appl. No.: 13/734,018

(22) Filed: Jan. 4, 2013

Related U.S. Application Data
(60) Provisional application No. 61/582,950, filed on Jan. 4, 2012.

(51) Int. Cl. A61F 13/00

(52) U.S. Cl. CPC A61F 13/00042
USPC 602/46; 602/43

(57) ABSTRACT

A device and method for removing moisture vapor from adjacent skin surfaces such as bariatric skin folds. The device may include a vapor permeable material, a spacer material, and an air mover that are configured to be inserted between adjacent skin surfaces. The device is inserted between adjacent skin surfaces and the air mover is operated to provide air flow through the spacer material.

### CROSS-REFERENCE TO RELATED APPLICATIONS

This application claims priority to U.S. Provisional Application No. 61/582,950 filed Jan. 4, 2012, the entire contents of which is incorporated by reference herein without disclaimer.

### FIELD OF THE INVENTION

The present disclosure relates generally to moisture removal devices and methods for bariatric skin folds that aid in the prevention, reduction, and/or treatment of decubitus ulcers and the transfer of moisture and/or heat from the body.

### BACKGROUND

Patients and other persons restricted to bed for extended periods incur the risk of forming decubitus ulcers. Decubitus ulcers (commonly known as bed sores, pressure sores, pressure ulcers, etc.) can be formed when blood supplying the capillaries below the skin tissue is interrupted due to external pressure against the skin. This pressure can be greater than the internal blood pressure within a capillary and thus, occlude the capillary and prevent oxygen and nutrients from reaching the area of the skin in which the pressure is exerted. Moreover, moisture and heat on and around the person can exacerbate ulcers by causing skin maceration, among other associated problems.

### SUMMARY

Exemplary embodiments of the present disclosure are directed to apparatus, systems and methods to aid in the prevention of decubitus ulcer formation and/or promote the healing of such ulcer formation.

In accordance with an exemplary embodiment, a method of removing moisture vapor from between adjacent skin surfaces of a bariatric person comprises inserting a moisture vapor removal device between adjacent skin surfaces of a bariatric person. The moisture removal device comprises a first layer comprising a vapor permeable material, a second layer comprising a vapor permeable material, a spacer material between the first layer and the second layer, and an air mover. The air mover is operated to provide air flow through the spacer material.

The air mover may provide air flow through the spacer material by pulling air through the spacer material toward the air mover.

The first layer may comprise an aperture and the air flow may be directed through the aperture toward the air mover.

The spacer material may comprise one of the following: open cell foam; natural or synthetic polymer particles, filaments, or strands; cotton fibers; polyester fibers; flexible metals and metal alloys; shape memory metals and metal alloys, and shape memory plastics.

The air flow may be less than about 2.0 cubic feet per minute at a differential pressure of less than about 6.0 mm H₂O.

The moisture removal device may comprise a base portion and one or more extensions from the base portion. The one or more extensions may be inserted between adjacent skin surfaces of the bariatric person.

The base portion may be placed on a chest area of the bariatric person.

The base portion may be placed between the bariatric person and a support surface.

In accordance with another exemplary embodiment, a device for removing moisture vapor comprises a first layer comprising a vapor permeable material, a second layer comprising a vapor permeable material, a spacer material between the first layer and the second layer; and an air mover adapted to provide air flow through the spacer material, wherein the device is adapted to be placed between adjacent skin surfaces of a bariatric person.

The first layer may further comprise an aperture. The aperture may be proximal to a first end of the device and the air mover may be proximal to a second end of the device.

The spacer material may comprise one of the following: open cell foam; natural or synthetic polymer particles, filaments, or strands; cotton fibers; polyester fibers; flexible metals and metal alloys; shape memory metals and metal alloys, and shape memory plastics.

The air mover may be adapted to pull air through the spacer material toward the air mover.

The device may comprise a length and a width and the width of the device may be approximately eight inches. The device may comprise a thickness of approximately one inch.

The air mover may be positioned such that the spacer material is between the air mover and the adjacent skin surfaces during use.

The device may comprise a base portion and one or more extensions from the base portion. The air mover may be located in the base portion and an aperture may be located within one of the one or more extensions. The base portion may be configured to be placed between a bariatric patient and a support surface and the one or more extensions may be configured to be placed between adjacent skin surfaces of the bariatric person.

The base portion may be configured to be placed on an abdominal region of the bariatric person and the one or more extensions may be configured to be placed between adjacent skin surfaces of the bariatric person.

The device may be adapted to be placed between adjacent skin surfaces in a chest area of a bariatric person.

The device may be adapted to be placed between adjacent skin surfaces in an abdominal area of a bariatric person.

The air mover may be configured to provide less than about 2.0 cubic feet per minute of air flow during operation at a differential pressure of less than about 6.0 mm H₂O.

### BRIEF DESCRIPTION OF THE DRAWINGS

While exemplary embodiments of the present invention have been shown and described in detail below, it will be clear to the person skilled in the art that changes and modifications may be made without departing from the scope of the invention. As such, that which is set forth in the following description and accompanying drawings is offered by way of illustration only and not as a limitation. The actual scope of the invention is intended to be defined by the following claims, along with the full range of equivalents to which such claims are entitled.

In addition, one of ordinary skill in the art will appreciate upon reading and understanding this disclosure that other variations for the invention described herein can be included within the scope of the present invention. For example, portions of the support system shown and described may be incorporated with existing mattresses or support materials.

In the following Detailed Description of Exemplary Embodiments, various features are grouped together in several embodiments for the purpose of streamlining the disclosure. This method of disclosure is not to be interpreted as reflecting an intention that exemplary embodiments of the invention require more features than are expressly recited in each claim. Rather, as the following claims reflect, inventive subject matter lies in less than all features of a single disclosed embodiment. Thus, the following claims are hereby incorporated into the Detailed Description of Disclosed Embodiments, with each claim standing on its own as a separate embodiment.

FIG. 1 illustrates a top view of a moisture vapor removal device according to a first exemplary embodiment of the present disclosure.

FIG. 2 illustrates a cross-sectional side view of the embodiment of FIG. 1.

FIG. 3 illustrates a top view of a bariatric patient.

FIG. 4 illustrates a top view of the embodiment of FIG. 1 during use with the bariatric patient of FIGS. 3.

FIG. 5 illustrates a top view of a moisture vapor removal device according to a second exemplary embodiment of the present disclosure.

FIG. 6 illustrates a cross-sectional side view of the embodiment of FIG. 5.

FIG. 7 illustrates a side view of the embodiment of FIG. 5 during use with a bariatric patient.

FIG. 8 illustrates a top view of a moisture vapor removal device according to a third exemplary embodiment of the present disclosure during use with a bariatric patient.

### DETAILED DESCRIPTION OF EXEMPLARY EMBODIMENTS

Exemplary embodiments of the present disclosure are directed to apparatus, systems and methods to aid in the prevention of decubitus ulcer formation and/or promote the healing of such ulcer formation. Exemplary embodiments may also aid in the prevention of other issues, including for example, odor, bacteria growth, or fungal growth. Existing systems may be used to prevent ulcer formation and/or healing decubitus ulcers through the use of a multi-layer cover sheet disposed between a patient and a supporting surface such as a mattress. Exemplary embodiments of the present disclosure can be utilized to aid in the removal of moisture, vapor, and heat between adjacent surfaces of a person's skin, for example in bariatric patients. Adjacent skin surfaces may be located, for example, in skin folds located in the breast, groin, or abdominal areas.

As explained in more detail below, exemplary embodiments may be placed between adjacent skin surfaces to reduce or remove moisture vapor from between the skin surfaces via air flow. In exemplary embodiments, an air mover may provide suction (or negative pressure) air flow through a vapor permeable outer layer and through a spacer material.

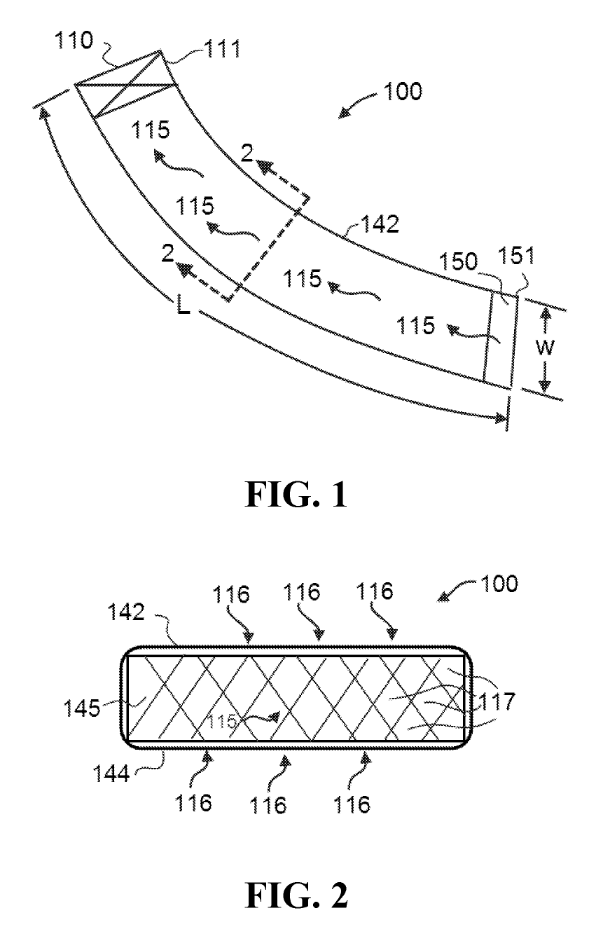

Referring now to FIGS. 1 and 2, an exemplary embodiment of a moisture vapor reduction device (MVRD) 100 comprises an air mover 110 proximal to a first end 111 and an aperture 150 proximal to a second end 151. In certain embodiments, aperture 120 may function as an air inlet that allows air flow from aperture 150 to air mover 110, which can provide suction air flow through MVRD 100. In other embodiments, aperture 150 may serve as an air outlet to allow air flow from air mover 110 to aperture 150 (e.g., when air mover is configured to provide positive pressure air flow through MVRD 100).

As shown in the section view of FIG. 2 (taken along line 2-2 in FIG. 1), MVRD 100 comprises a first layer 142, a second layer 144, and a spacer material 145 between the first and second layers 142, 144. In FIG. 2, first and second layers 142, 144 are shown slightly separated from spacer material 145 for purposes of clarity. It is understood that the Figures are not to scale, and that in actual embodiments first and second layers 142, 144 may be in contact with spacer material 145. In this embodiment, MVRD 100 comprises a length L extending between first end 111 and second end 151, and a width W extending perpendicular to length L. In certain exemplary embodiments, the width W is approximately eight inches and the length L is approximately twenty-four inches. In certain embodiments, MVRD may comprise a thickness T of approximately 1.5 inches, 1.25 inches, 1.0 inches, 0.75 inches, 0.5 inches or less.

In particular exemplary embodiments, spacer material 145 may comprise an open-cell foam and first and second layers 142, 144 may comprise a vapor permeable, liquid impermeable material (including for example, nylon taffeta or Gore-Text). As used in this disclosure, the term “spacer material” (and related terms) should be construed broadly to include any material that includes a volume of air within the material and allows air to move through the material. In exemplary embodiments, spacer materials allow air to flow through the material when the material is placed between adjacent skin surface, including e.g, skin folds of bariatric patients. Examples of such spacer materials include open cell foam, polymer particles, and a material sold by Tytex under the trade name AirX™.

In certain exemplary embodiments, air mover 110 is configured to draw air through spacer material 145. In the embodiment shown, air flow 115 enters spacer material 145 at an area distal from air mover 110. Air flow 115 can then move from aperture 150, through spacer material 145 and exit via air mover 110.

In certain exemplary embodiments, air mover 110 is a 12 volt DC, 40 mm box fan such as a Sunon KDE 1204 PKBX-8. By utilizing an air mover such as the Sunon model (or other similarly-sized devices), air mover 110 can be placed integral with spacer material 145, allowing for a more compact overall design of MVRD 100. In certain exemplary embodiments, air mover 110 is a centrifugal 12 volt (nominal) DC fan manufactured by Panasonic under the part number FAL5F12LL. This particular air mover is approximately 3 inches wide by 3 inches tall by 1.1 inches thick and weighs approximately 3.5 ounces. This air mover also produces a maximum air flow of approximately 8.8 cfm and maximum air pressure of approximately 6.2 mmH2O at a nominal 12 volts. During operation, the air flow will be reduced as the pressure across the air mover is increased. Exemplary embodiments using this air mover typically have an air flow of approximately 1.0 to 2.0 cfm during operation. The Panasonic FAL5F12LL air mover also creates low noise levels (30.0 dB-A, according to the manufacturer's specifications).

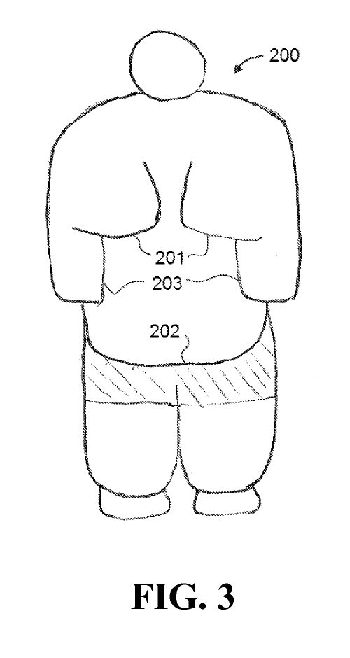

Referring now to FIG. 3, a bariatric patient 200 is shown with areas that include adjacent skin surfaces that may be prone to developing decubitus ulcers and other skin integrity issues. For example, chest area 201, abdominal area 202 and arm-torso area 203 are exemplary of such areas. It is understood that other areas on bariatric patients can be prone to developing decubitus ulcers and are suitable for use with MVRD 100.

Referring now to FIG. 3, MVRD 100 is shown placed between adjacent skin surfaces in chest area 201. In this embodiment, MVRD 100 is shown such that first end 111 and air mover 110 are between (and covered by) the adjacent skin surfaces of chest area 201. In this embodiment, aperture 150 is not between the adjacent skin surfaces and is exposed to the outside environment.

During operation, air mover 110 provides a suction air flow and draws or pulls air from the outside environment, through aperture 150, spacer material 145 and exits through air mover 110. In certain embodiments, air mover 110 may be positioned such that spacer material 145 is between air mover 110 and the adjacent skin surfaces in chest area 201. In other embodiments, MVRD 100 may comprise a cage-like structure around air mover 110 to provide spacing between air mover 110 and the adjacent skin surfaces. Such spacing between air mover 110 and the adjacent skin surfaces can allow for increased air flow through MVRD 100.

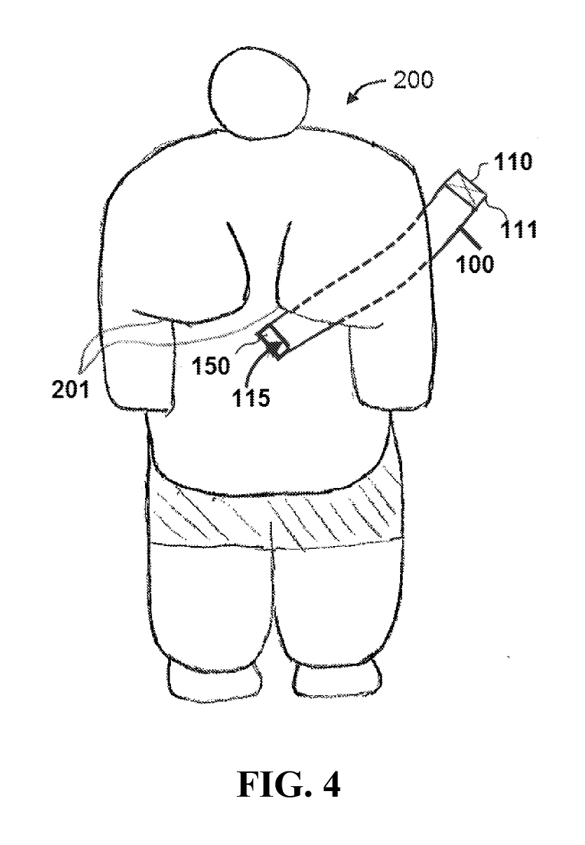

Referring now to FIGS. 2-4, during use moisture vapor 116 (from chest area 201) can pass through first and second layers 142, 144 and into air pockets 117 within spacer material 145. Moisture vapor will continue to transfer to air pockets 117 within spacer material 145 while air pockets 117 are at a lower relative humidity than the space within chest area 201 (e.g. between the adjacent skin surfaces of chest area 201). As the relative humidity of air pockets 117 increases and approaches the relative humidity of chest area 201, the transfer rate of the moisture vapor will decrease. It is therefore desirable to maintain a lower relative humidity of air pockets 117 within spacer material 145 than the relative humidity of the space within chest area 201.

As moisture vapor 116 is transferred to air pockets air pockets 117, it is desirable to remove moisture vapor from air pockets 117 and lower the relative humidity of the air within spacer material 145. By removing moisture vapor from the air within spacer material 145, the transfer rate of moisture vapor from chest area 201 can be maintained at a more uniform level. The use of air mover 110 to create air flow 115 through MVRD 100 can assist in removing moisture vapor from air pockets 117 and the air within spacer material 145.

For example, air flow 115 can introduce air from the surrounding environment (which is at a lower relative humidity than the space in chest area 201) to air pockets 117. In the embodiments shown, air flow 115 enters MVRD 100 through aperture 150 and flows through air pockets 117 within spacer material 145 and assists in removing moisture vapor 116 from air pockets 117. This lowers the relative humidity of air pockets 117 and allows the transfer rate of moisture vapor 116 to be maintained over time. Air flow 115 can also reduce the temperature within chest area 201, as air is pulled into MVRD 100 that is a lower temperature than the air within chest area 201.

The likelihood of decubitus ulcers or other damaging skin conditions forming in chest area 201 is reduced by providing a reduced temperature and more uniform transfer rate of moisture vapor 116 from chest area 201. It is understood that MVRD 100 may be used in a manner similar to that described above to provide moisture vapor transfer from abdominal area 202, arm-torso area 203, or any other area with adjacent skin surfaces.

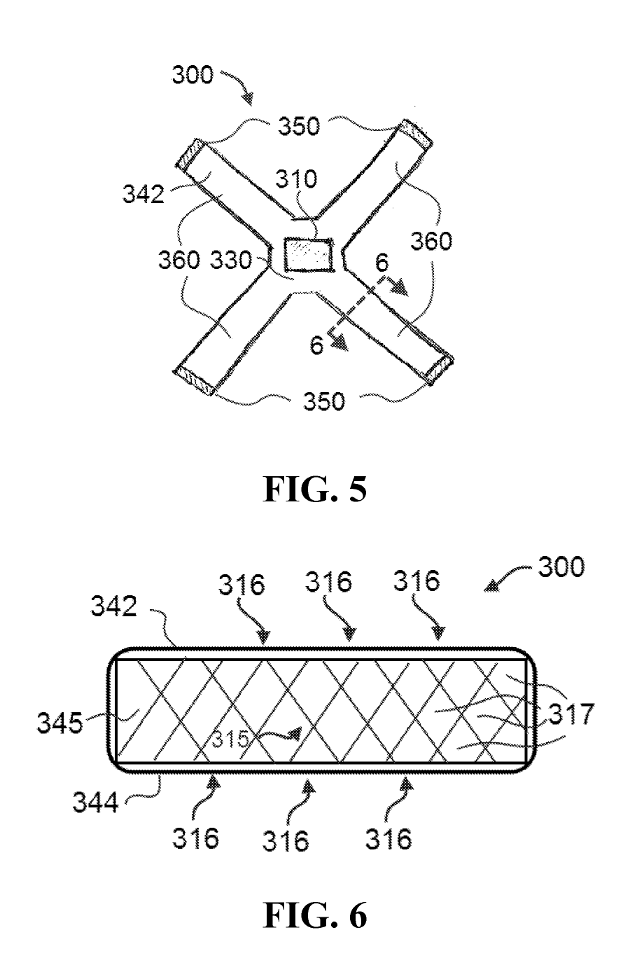

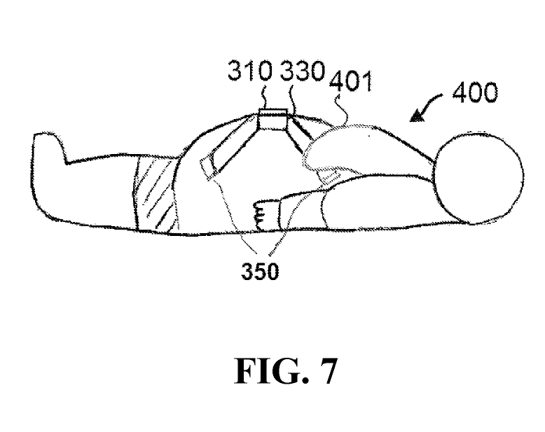

Referring now to FIGS. 5-7, a second exemplary embodiment of an MVRD 300 comprises a central portion 330 with an air mover 310. MVRD 300 also comprises a plurality of extensions 360, each with an aperture 350 that is distal from central portion 330. FIG. 6 illustrates a cross-section of MVRD taken along line 6-6 of FIG. 5. FIG. 7 illustrates MVRD during use with a bariatric patient 400.

MVRD 300 is configured similar to MVRD 100 described above. However, with MVRD 300 each extension 360 may be placed between separate regions with adjacent skin surfaces. In the embodiment shown in FIG. 7, an extension 360 is placed between skin surfaces in a chest area 401. It is understood that the remaining extensions 360 may be placed between adjacent skin surfaces in other regions. In this embodiment, air mover 310 is centrally located and can draw or pull air flow 315 (shown in FIG. 6) from apertures 350 to air mover 310. It is understood that in other exemplary embodiments, air mover 310 may be configured to push air flow 315 from air mover 310 toward aperture 350.

Referring now to FIG. 6, the cross-section view of MVRD 300 illustrates a configuration similar to that of the previously-described embodiment. MVRD 300 comprises a first layer 342, a second layer 344, and a spacer material 345 between the first and second layers 342, 344. In FIG. 6, first and second layers 342, 344 are shown slightly separated from spacer material 345 for purposes of clarity. It is understood that the Figures are not to scale, and that in actual embodiments first and second layers 342, 344 may be in contact with spacer material 345. In this embodiment, first and second layers 342, 344 and spacer material 345 may be comprised of the materials disclosed in the description of the previously-described embodiment.

In addition, MVRD 300 operates in a manner similar to MVRD 100. During operation, moisture vapor 316 (from chest area 401) can pass through first and second layers 342, 344 and into air pockets 317 within spacer material 345. Moisture vapor 316 will continue to transfer to air pockets 317 within spacer material 345 while air pockets 317 are at a lower relative humidity than the space within chest area 401. As explained in the previously-described embodiment, the use of air mover 310 to create air flow 315 through MVRD 300 can assist in removing moisture vapor from within spacer material 345. Consequently, the use of MVRD 300 can maintain the transfer rate of moisture vapor from chest area 401 and reduce the temperature. The likelihood of decubitus ulcers or other damaging skin conditions forming in chest area 401 is reduced by providing a reduced temperature and more uniform transfer rate of moisture vapor 316 from chest area 401.

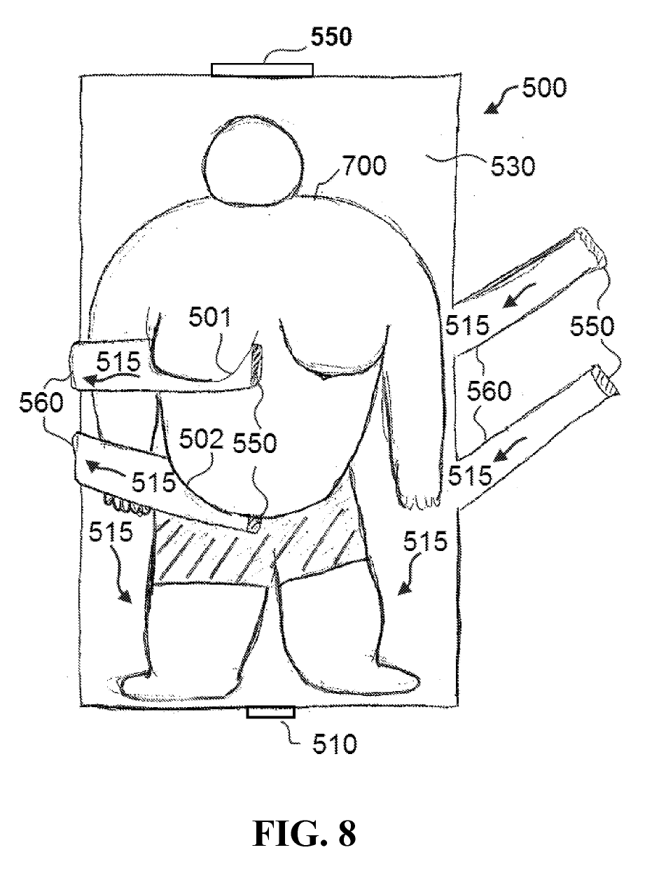

Referring now to FIG. 8, another exemplary embodiment illustrates an MVRD 500 comprising a base portion 530 with an air mover 510. MVRD 500 also comprises a plurality of extensions 560, each with an aperture 550 that is distal from central portion 530.

MVRD 500 is configured similar to MVRD 100 and MVRD described above. However, MVRD 500 comprises base portion 530 adapted to be placed between a bariatric patient 700 and a support surface, including e.g. a mattress. In this exemplary embodiment, base portion 530 is constructed in a manner equivalent to the previously-described embodiments. For example, base portion 530 comprises a spacer material between two vapor permeable layers. However, instead of being placed between adjacent skin surfaces, base portion is placed between the patient's body and the supporting surface.

Similar to MVRD 300, each extension 560 of MVRD 500 may be placed between separate regions of bariatric patient 700 with adjacent skin surfaces. In the embodiment shown in FIG. 8, one extension 560 is placed between skin surfaces in a chest area 501, while another extension 560 is placed between skin surfaces in an abdominal area 502. It is understood that the remaining extensions 560 may be placed between adjacent skin surfaces in other regions. In this embodiment, air mover 510 is located in base portion 530 and can draw or pull air flow from apertures 550 to air mover 510. In certain embodiments, base portion 530 may also comprise apertures distal from air mover 510 to also admit air into MVRD 500. It is understood that in other exemplary embodiments, air mover 510 may be configured to push air flow 515 from air mover 510 toward aperture 550.

In various exemplary embodiments, an MVRD may include a number of layers. Each layer may be formed of a number of different materials that exhibit various properties. These properties may include the level of friction or shear of a surface, the permeability of a vapor, a gas, a liquid, and/or a solid, and various phases of the vapor, the gas, the liquid, and the solid, and other properties.

In certain exemplary embodiments, an MVRD may be configured such that the air mover is external to the MVRD and is coupled to MVRD with appropriate conduit or other coupling members. In exemplary embodiments with an external air mover, the air mover may be conveniently mounted in an accessible location, such as the foot board of a bed frame supporting the cover sheet and support mattress.

As one of ordinary skill in the art will appreciate, vapor and air can carry organisms such as bacteria, viruses, and other potentially harmful pathogens. As such, in certain embodiments of the present disclosure, one or more antimicrobial devices, agents, etc., can be provided to prevent, destroy, mitigate, repel, trap, and/or contain potentially harmful pathogenic organisms including microbial organisms such as bacteria, viruses, mold, mildew, dust mites, fungi, microbial spores, bioslimes, protozoa, protozoan cysts, and the like, and thus, remove them from air and from vapor that is dispersed and removed from the patient and from the environment surrounding the patient. Examples of antimicrobial devices can include mechanical devices such as filters, energy devices such as ultraviolet light sources, and chemical agents such as antimicrobial coatings. Other antimicrobial devices and agents are also contemplated. In addition, in various embodiments, the MVRD can include various layers having antimicrobial activity. In some embodiments, for example, the spacer material can include particles, fibers, threads, etc., formed of silver and/or other antimicrobial agents.

In various exemplary embodiments, the spacer material can be formed of various materials, and can have a number of configurations and shapes, as described herein. In some embodiments, the material is flexible. In such exemplary embodiments, the flexible material can include properties that resist compression, such that when the flexible material is subjected to the weight of a patient or skin fold lying on the MVRD, the flexible material has a tendency to return toward its original shape, and thereby impart a supportive function to the MVRD. The flexible material can also include a property that allows for lateral movement of air through the flexible material even under compression.

Examples of materials that can be used to form the spacer material can include, but are not limited to, natural and synthetic polymers in the form of particles, filaments, strands, foam (e.g., open cell foam), among others, and natural and synthetic materials such as cotton fibers, polyester fibers, and the like. Other materials can include flexible metals and metal alloys, shape memory metals and metal alloys, and shape memory plastics. These materials can include elastic, super elastic, linear elastic, and/or shape memory properties that allow the flexible material to flex and bend and to form varying shapes under varying conditions (e.g., compression, strain, temperature, etc.).

In various exemplary embodiments the spacer material can be chemically attached to the outer layers (e.g., the first and second layers of the previously-described embodiments) through the use of adhesives, and the like, and/or mechanically attached through the use of fasteners such as stitches, clasps, hook and loop, and the like, and/or physically attached through the use of welds, such as RF welds and related methods.

In some exemplary embodiments, the first and second layers can be fastened together by stitching, buttons and/or hook and loop fasteners (i.e., VELCRO®) or the like. In other exemplary embodiments, the first and second layers may be fastened together by welding them together along their perimeters using high frequency radio energy (i.e., RF welding) or ultrasonic energy (i.e., ultrasonic welding). Other forms of welding are also contemplated.

In various exemplary embodiments, an MVRD can be a one-time use device or a multi-use device. As used herein, a one-time use device is a device for single-patient use applications that is formed of a vapor, air, and liquid permeable material that is disposable and/or inexpensive and/or manufactured and/or assembled in a low-cost manner and is intended to be used for a single patient over a brief period of time, such as an hour(s), a day, or multiple days. As used herein, a multi-use device is a device for multi-patient use that is generally formed of a vapor permeable, liquid impermeable and air permeable or air impermeable material that is re-usable, washable, can be disinfected using a variety of techniques (e.g., autoclaved, bleach, etc.) and generally of a higher quality and superior in workmanship than the one-time use device and is intended to be used by one or more patients over a period of time such as multiple days, weeks, months, and/or years. In various exemplary embodiments, manufacturing and/or assembly of a multi-use device can involve methods that are more complex and more expensive than one-time use devices. Examples of materials used to form one-time use device can include, but are not limited to, non-woven papers. Examples of materials used to form re-usable device can include, but are not limited to, Gore-Tex®, and urethane laminated to fabric.

1. A method of removing moisture vapor from between adjacent skin surfaces of a bariatric person, the method comprising:
inserting a moisture vapor removal device between adjacent skin surfaces of a bariatric person, wherein the moisture removal device comprises:
a first layer comprising a vapor permeable material;
a second layer comprising a vapor permeable material;
a spacer material between the first layer and the second layer; and
an air mover; and
operating the air mover to provide air flow through the spacer material.

2. The method of claim 1, wherein operating the air mover to provide air flow through the spacer material comprises pulling air flow through the spacer material toward the air mover.

3. The method of claim 1, wherein the first layer comprises an aperture and wherein the air flow is directed through the aperture toward the air mover.

4. The method of claim 1, wherein the spacer material comprises one of the following: open cell foam; natural or synthetic polymer particles, filaments, or strands; cotton fibers; polyester fibers; flexible metals and metal alloys; shape memory metals and metal alloys, and shape memory plastics.

5. The method of claim 1, wherein the air flow is less than about 2.0 cubic feet per minute at a differential pressure of less than about 6.0 mm H₂O.

6. The method of claim 1 wherein:
the moisture removal device comprises a base portion and one or more extensions from the base portion; and
inserting the moisture removal device between adjacent skin surfaces of a bariatric person comprises inserting the extension between adjacent skin surfaces of the bariatric person.

7. The method of claim 6, wherein the base portion is placed on a chest area of the bariatric person.

8. The method of claim 6, wherein the base portion is placed between the bariatric person and a support surface.

9. A device for removing moisture vapor comprising:
a first layer comprising a vapor permeable material;
a second layer comprising a vapor permeable material;
a spacer material between the first layer and the second layer; and
an air mover adapted to provide air flow through the spacer material, wherein the device is adapted to be placed between adjacent skin surfaces of a bariatric person.

10. The device of claim 9, wherein the first layer further comprises an aperture.

11. The device of claim 10, wherein the aperture is proximal to a first end of the device and wherein the air mover is proximal to a second end of the device.

12. The device of claim 9, wherein the spacer material comprises one of the following: open cell foam; natural or synthetic polymer particles, filaments, or strands; cotton fibers; polyester fibers; flexible metals and metal alloys; shape memory metals and metal alloys, and shape memory plastics.

13. The device of claim 9, wherein the air mover is adapted to pull air through the spacer material toward the air mover.

14. The device of claim 9, wherein the device comprises a length and a width and wherein the width of the device is approximately eight inches.

15. The device of claim 9, wherein the device comprises a thickness of approximately one inch.

16. The device of claim 9, wherein the air mover is positioned such that the spacer material is between the air mover and the adjacent skin surfaces during use.

17. The device of claim 9, wherein the device comprises a base portion and one or more extensions from the base portion.

18. The device of claim 17, wherein the air mover is located in the base portion.

19. The device of claim 17, wherein the air mover is located in the base portion and an aperture is located within one of the one or more extensions.

20. The device of claim 17, wherein the base portion is configured to be placed between a bariatric patient and a support surface and wherein the one or more extensions are configured to be placed between adjacent skin surfaces of the bariatric person.

21. The device of claim 17, wherein the base portion is configured to be placed on an abdominal region of the bariatric person and wherein the one or more extensions are configured to be placed between adjacent skin surfaces of the bariatric person.

22. The device of claim 9, wherein the device is adapted to be placed between adjacent skin surfaces in a chest area of a bariatric person.

23. The device of claim 9, wherein the device is adapted to be placed between adjacent skin surfaces in an abdominal area of a bariatric person.

24. The device of claim 9, wherein the air mover is configured to provide less than about 2.0 cubic feet per minute of air flow during operation at a differential pressure of less than about 6.0 mm H₂O.
